(19) United States
(12) Patent Application Publication
(10) Pub. No.: US 2026/0000281 A1
(43) Pub. Date: Jan. 1, 2026

(54) Apparatuses and Methods for a Tubular Concentric Tube Continuum Manipulator

(71) Applicant: EndoTheia, Inc., Nashville, TN (US)

(72) Inventors: Joshua Gafford, Nashville, TN (US); Scott Webster, Nshville, TN (US); Patrick Anderson, Nashville, TN (US); Caleb Rucker, Knoxville, TN (US); Jake Childs, Nashville, TN (US)

(21) Appl. No.: 19/250,804

(22) Filed: Jun. 26, 2025

Related U.S. Application Data
(60) Provisional application No. 63/664,717, filed on Jun. 26, 2024.

(51) Int. Cl. A61B 1/008; A61B 1/00; A61B 1/04; A61B 1/06; A61B 34/30

(52) U.S. Cl. CPC A61B 1/008; A61B 1/00137; A61B 1/042; A61B 1/06; A61B 34/30; A61B 2034/301

(57) ABSTRACT

The present disclosure provides for a visual inspection apparatus. The apparatus may include a concentrically nested tube assembly having a first tube, a second tube, and a third tube. In some embodiments, the first tube includes a first deflectable section, the second tube includes a second deflectable section, and the third tube includes a third deflectable section. The first, second, and third deflectable sections may be selectively weakened portions of the first, second, and third tubes. The first and second tubes may be joined at a location distal to the first and second deflectable sections. The tube assembly may be actuable to form a first bend by relative axial translation between the first tube and the second tube, and the tube assembly may be actuable to form a second bend by relative axial transaction between the second tube and the third tube. Such bending may be co-planar or non-co-planar.

### CROSS-REFERENCES TO RELATED APPLICATIONS

This application is a non-provisional of and claims benefit of and priority to U.S. Provisional Application No. 63/664,717, filed Jun. 26, 2024, entitled “APPARATUSES AND METHODS FOR A TUBULAR CONCENTRIC TUBE CONTINUUM MANIPULATOR,” which is hereby incorporated by reference in its entirety.

### STATEMENT REGARDING FEDERALLY SPONSORED RESEARCH OR DEVELOPMENT

Not Applicable.

### REFERENCE TO SEQUENCE LISTING OR COMPUTER PROGRAM LISTING APPENDIX

Not Applicable.

### BACKGROUND

The present disclosure relates to endoscopic surgical procedures. More particularly, the present disclosure relates to enhanced apparatuses and methods of manipulating tube assemblies for endoscopic surgical procedures.

Endoscopic surgical procedures often require navigation of tortuous pathways. Accordingly, endoscopes may need to bend in multiple, non-coplanar directions. However, conventional systems are typically configured to bend in a single, co-planar direction. Thus, conventional systems may need to be removed, reinserted, and advanced into bodily cavities multiple times in order to navigate particular pathways.

It would be advantageous to provide an ureteroscope, or components thereof, that provides enhanced steerable functionality, particularly as it pertains to steering in multiple, non-coplanar directions.

### BRIEF SUMMARY

This Brief Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

The present disclosure provides for a visual inspection apparatus (e.g., an endoscopic apparatus, a borescope apparatus, etc.). The apparatus may include a tube assembly having a first tube, a second tube concentrically nested within the first tube, and a third tube concentrically nested within the second tube.

In some embodiments, the first tube includes a first deflectable section, the second tube includes a second deflectable section, and the third tube includes a third deflectable section. The first, second, and third deflectable sections may be selectively weakened portions of the first, second, and third tubes. The first and second tubes may be joined at a location distal to the first and second deflectable sections,

In some embodiments, the second tube includes a fourth deflectable section, and the third tube includes a fifth deflectable section. The fourth and fifth deflectable sections may be selectively weakened portions of the second and third tubes. The first and second tubes are joined at a location distal to the first and second deflectable sections.

The tube assembly may be actuable to form a first bend by relative axial translation between the first tube and the second tube, and the tube assembly may be actuable to form a second bend by relative axial transaction between the second tube and the third tube. Such bending may be co-planar or non-co-planar.

Thus, specific embodiments described herein consist of three tubes, an outer tube, a second tube, and a third tube, which have each been mechanically configured to exhibit preferential bending in one or more planes, and are attached to each other at various points along the tubes. By linearly translating and/or rotating the tubes with respect to each other at the proximal ends, the tubes can be configured to articulate at the distal end along various degrees of freedom. Such an apparatus may have utility in rigid or flexible endoscopic robotic systems. In a generalized sense, a set of N tubes can be used to actuate N-1 bending degrees of freedom.

Such an apparatus may have utility in the field of flexible surgical robotics. A specific embodiment is proposed where the manipulator system is used to create a robotically controlled video endoscope for a flexible endoluminal robotic system. Such a system may also have utility in industrial applications, for example, in the creation of articulating boroscopes for industrial inspection.

Numerous other objects, advantages and features of the present disclosure will be readily apparent to those of skill in the art upon a review of the following drawings and description of a preferred embodiment.

### BRIEF DESCRIPTION OF THE DRAWINGS

FIG. 1 is an exploded view of a visual inspection apparatus, according to some embodiments of the present disclosure.

FIG. 2 is a perspective view of the apparatus of FIG. 1 being actuated to form a bend, according to some embodiments of the present disclosure.

FIG. 3 is a perspective view of a visual inspection apparatus, according to further embodiments of the present disclosure.

FIG. 4 is a detailed perspective view of a visual inspection apparatus, according to further embodiments of the present disclosure.

FIG. 5 is a perspective view of the apparatus of FIG. 3 being actuated to form a first bend, according to some embodiments of the present disclosure.

FIG. 6 is a perspective view of the apparatus of FIG. 3 being actuated to form a second bend, according to some embodiments of the present disclosure.

FIG. 7 is a perspective view of the apparatus of FIG. 3, rotated to form bends of multiple degrees of freedom, according to some embodiments of the present disclosure.

FIG. 8 is a perspective view of the apparatus of FIG. 7 being actuated to form bends of multiple degrees of freedom, according to some embodiments of the present disclosure.

FIG. 9 is a perspective view of the apparatus of FIG. 7 being actuated to perform a panning movement, according to some embodiments of the present disclosure.

FIG. 10 is a perspective view of the apparatus of FIG. 7 being actuated to perform a panning movement, according to further embodiments of the present disclosure.

FIG. 11 is a perspective view of the tube of the apparatus of FIG. 1 with a decoupling segment, according to some embodiments of the present disclosure.

FIG. 12 is perspective view of the apparatus of FIG. 9 with the decoupling segment of FIG. 11, according to some embodiments of the present disclosure.

FIG. 13 is a perspective view of the apparatus of FIG. 1 with a videoscope, according to some embodiments of the present disclosure.

FIG. 14 is a detailed view of the videoscope of FIG. 13, according to some embodiments of the present disclosure.

FIG. 15 is an exploded view of a visual inspection apparatus, according to further embodiments of the present disclosure.

FIG. 16 is a side view of the apparatus of FIG. 1 with a transmission section, according to some embodiments of the present disclosure.

FIG. 17 is a perspective view of an endoscopic system including the apparatus of FIG. 4, according to some embodiments of the present disclosure.

FIG. 18 is a side view of the system of FIG. 17, according to some embodiments of the present disclosure.

FIG. 19 is a side view of the system of FIG. 17 being deployed, according to some embodiments of the present disclosure.

FIG. 20 is a side view of the system of FIG. 17 being deployed, according to further embodiments of the present disclosure.

FIG. 21 is a side view of the system of FIG. 17 with the apparatus being actuated to form a bend, according to some embodiments of the present disclosure.

### DETAILED DESCRIPTION

While the making and using of various embodiments of the present invention are discussed in detail below, it should be appreciated that the present invention provides many applicable inventive concepts that are embodied in a wide variety of specific contexts. The specific embodiments discussed herein are merely illustrative of specific ways to make and use the invention and do not delimit the scope of the invention. Those of ordinary skill in the art will recognize numerous equivalents to the specific apparatus and methods described herein. Such equivalents are considered to be within the scope of this invention and are covered by the claims.

The present disclosure provides for a visual inspection apparatus (“apparatus”) 10. As a first example, the apparatus 10 may be an endoscopic apparatus. As a second example, the apparatus 10 may be a borescope or borescopic apparatus. Depending on the implementation, the apparatus 10 may be considered a system, and thus may alternatively be considered a system 10.

A discussed in greater detail below, the apparatus 10 may be a flexible continuum manipulator. The apparatus 10 may include a number of concentrically-nested tubes with various sequences of overlapping active and passive joints, which can be individually actuated to provide decoupled steerability of multiple degrees of freedom at a distal end thereof. For instance, such actuation may be achieved via an agonist/antagonist actuation methodology, in which pairs of tubes are configured to bend at their distal end by linearly translating the tubes with respect to each other at their proximal ends. In particular, this may be achieved through a selective weakening of each of the tubes at their distal ends in a manner that offsets their neutral axes away from the centerline of the tubes, and then joining the two tubes at their tips (e.g., at their distal tips on the aforementioned distal ends).

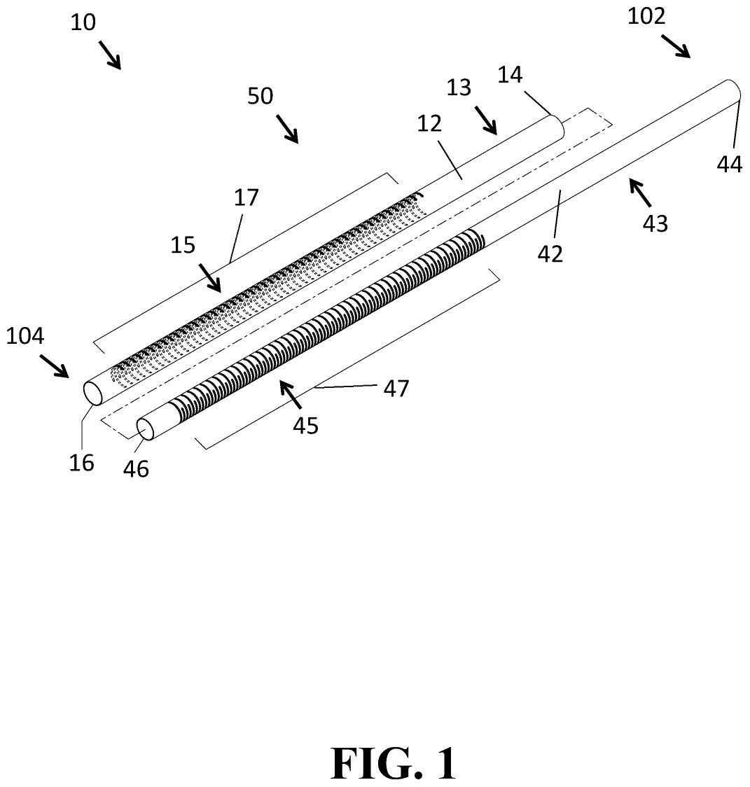

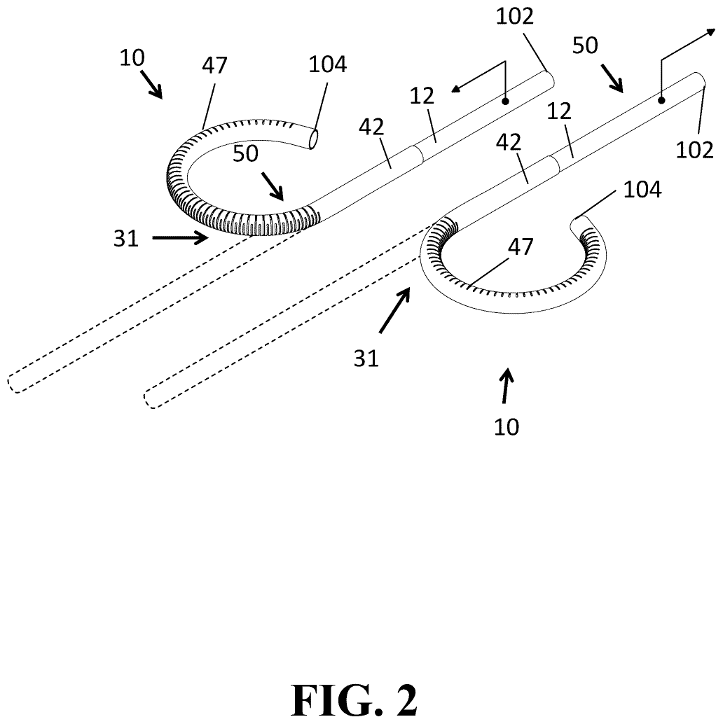

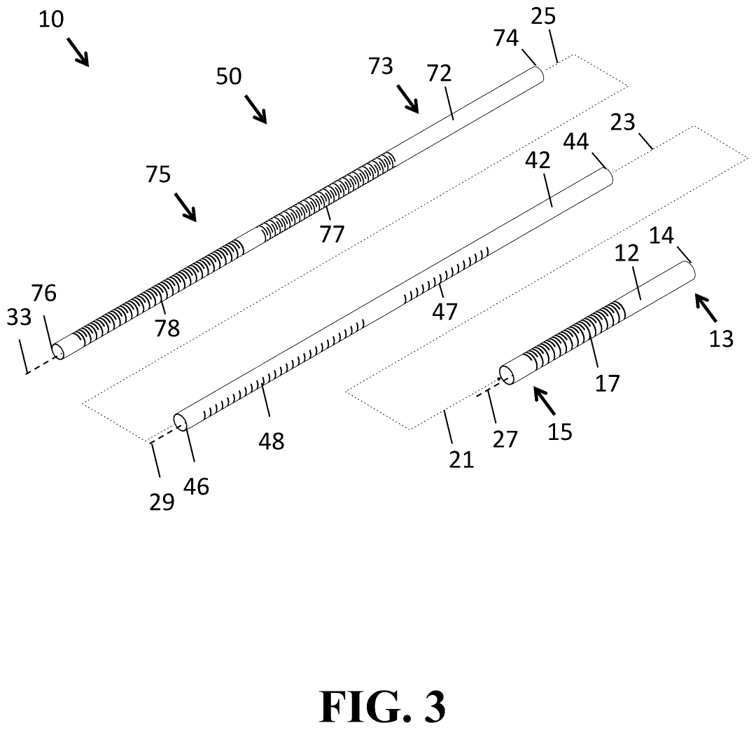

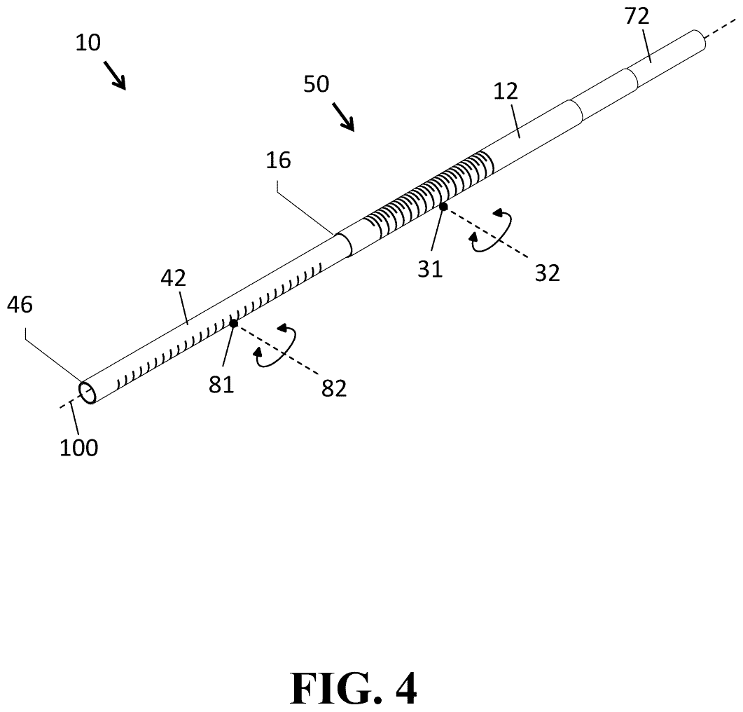

Referring now to FIGS. 1-2, an apparatus 10 for endoscopic surgery (apparatus) is shown, according to some embodiments of the present disclosure. FIG. 1 depicts an exploded view of the apparatus 10, while FIG. 2 depicts multiple implementations of the apparatus 10 being actuated to form a bend. The apparatus 10 may include a first (proximal) tip 102 and a second (distal) tip 104. In some embodiments, the apparatus 10 includes a first (inner) tube 12 and a second (middle or outer) tube 42. As shown with additional reference to FIGS. 3 and 4, the apparatus 10 may include a third (outer) tube 72. Each of the first, second, and third tubes 12, 42, 72 may be made of any suitable material including but not limited to any number of biocompatible metals (e.g., Nitinol, Stainless Steel, Titanium, etc.) and biocompatible plastics (e.g., polyimide, Nylon, PEEK, PEBAX).

The first and second tubes 12, 42, or the first, second, and third tubes 12, 42, 72 may be referred to herein as a tube assembly 50. Depending on the implementation, the apparatus 10 may include the tube assembly 50, or otherwise itself be considered a tube assembly. Thus, the apparatus 10 may include the tube assembly 50, which may include the first tube 12, the second tube 42 concentrically nested within the first tube 12, and the third tube 72 concentrically nested within the second tube 42.

The first tube 12 may define a first longitudinal axis 21 (shown with additional reference to FIG. 3) and include a first proximal end 13 terminating at a first proximal tip 14, a first distal end 15 terminating at a first distal tip 16, and a first slot pattern (e.g., a micromachined slot pattern) 17 defined between the first proximal tip 14 and the first distal tip 16. As generally discussed herein, the first slot pattern 17 may be considered a first deflectable section 17, which may be a selectively weakened portion of the first tube 12.

The second tube 42 may define a second longitudinal axis 23 (shown with additional reference to FIG. 3) and include a second proximal end 43 terminating at a second proximal tip 44, a second distal end 45 terminating in a second distal tip 46, and a second slot pattern 47 defined between the second proximal tip 44 and the second distal tip 46. As generally discussed herein, the second slot pattern 47 may be considered a second deflectable section 47, which may be a selectively weakened portion of the second tube 42.

As shown with additional reference to FIG. 3, the third tube 72 may define a third longitudinal axis 25 and include a third proximal end 73 terminating at a third proximal tip 74, a third distal end 75 terminating in a third distal tip 76, and a third slot pattern 77 defined between the third proximal tip 74 and the third distal tip 76. As generally discussed herein, the third slot pattern 77 may be considered a third deflectable section 77, which may be a selectively weakened portion of the third tube 72. Accordingly, the first tube 12 may include the first deflectable section 42, the second tube 42 may include the second deflectable section 47, and the third tube may includes the third deflectable section 77, the first, second, and third deflectable sections 17, 47, 77 being selectively weakened portions of the first, second, and third tubes 12, 42, 72.

As discussed herein, the first, second, and third distal ends 15, 45, 75 may be considered “bending sections.” Depending on the implementation, the first, second, and third slot patterns 17, 47, 77 may be positioned on the respective first, second, and third distal ends 15, 45, 75. In this sense, rather than the first, second, and third slot patterns 17, 47, 77, the first, second, and third ends 15, 45, 75 may be considered the aforementioned deflectable sections, and may thus be considered the first, second, and third deflectable sections 15, 45, 75.

The second tube 42 may be disposed in (e.g., housed in, nested in, inserted within, etc.) the first tube 12. For instance, the second tube 42 may be concentrically nested within the first tube 12, thus forming the tube assembly 50 mentioned above, according to some embodiments. As discussed in greater detail below, the third tube 72 may be disposed in the second tube 42. Thus, the third tube 72 maybe concentrically nested within the second tube 42, which may be concentrically nested within the first tube 12, thus forming the tube assembly 50 mentioned above, according to further embodiments.

Accordingly, the apparatus 10 may be a flexible continuum manipulator. The apparatus 10 may include a number of concentrically-nested tubes (e.g., the first, second, and third tubes 12, 42, 72) with various sequences of overlapping active and passive joints (e.g., the first, second, and third distal ends 15, 45, 75, the “bending sections”), which can be individually actuated to achieve decoupled steerability of multiple degrees of freedom at the second end 104 of the apparatus 10. As suggested above, such concentrically-nested tubes may further include various sequences of overlapping slot patterns (e.g., the first, second, and third slot patterns 17, 47, 77).

In some embodiments, actuation is achieved via an agonist/antagonist actuation methodology, in which the first and second tubes 12, 42 are configured to bend at the first and second distal ends 15, 45 (respectively) by linearly translating the first and second tubes 12, 42 with respect to each other at the first and second proximal ends 13, 43. For instance, such bending may be achieved through a selective weakening of each of the first and second tubes 12, 42 near their first and second distal ends 14, 44 (e.g., the first and second slot patterns 15, 45) in a manner that offsets their neutral axes away from the first and second longitudinal axes 21, 23 of the first and second tubes 12, 42 (respectively), and then joining the first and second tubes 12, 42 at the first and second distal tips 16, 46 (e.g., via a weld joint). For example, and with additional reference to FIG. 3, selective weaking of each of the first, second, third tubes 12, 42, 72 may result in first, second, and third neutral axes 27, 29, 33 (respectively), which may be offset from the respective first, second, and third longitudinal axes 21, 23, 25. Of course, when assembled as discussed herein, the first, second, and third neutral axes 27, 29, 33 (or a combination of the foregoing, such as the first and second neutral axes 27, 29) may define a combined neutral axis of the overall tube assembly 50 or apparatus 10 that is understood as being offset from the centerline axis 100.

As shown with particular reference to FIG. 2, the aforementioned actuation may form a first bending joint 31 (e.g., a single, bi-directional planar or “co-planar” bending joint). In this sense, the first bending joint 31 may be created with two concentrically-nested tubes (e.g., the first and second tubes 12, 42) which have each been modified such that a portion of each tube's neutral axis (e.g., the first and second neutral axes 27, 29) is offset from their respective longitudinal axes (e.g., the first and second longitudinal axes 21, 23), or in other words a centerline (e.g., longitudinal, tubular, etc.) axis 100 of the tube or tube assembly 50 of the apparatus 10. In this sense, the apparatus 10 may include or define the centerline axis 100.

As shown, the first bending joint 31 may be formed by the first and second tubes 12, 42. As discussed in greater detail below, in such embodiments where the apparatus 10 includes the third tube 72, the apparatus 10 may be similarly configured to form a second bending joint 81 in addition to the first bending joint 31. The second bending joint 81 may be formed by the second and third tubes 42, 72.

As discussed herein, the first bending joint 31 may be a portion of the tube assembly 50 configured to form a first bend, and the second bending joint 81 may be a portion of the tube assembly 50 configured to form a second bend. Thus, the tube assembly 50 may be actuable to form the first bend (e.g., a deflection at the first bending joint 31) by relative axial translation between the first and second tubes 12, 42; and the tube assembly 50 may be actuable to form the second bend (e.g., a deflection at the first second joint 81) by relative axial translation between the second and third tubes 42, 72.

As mentioned above, the first and second tubes 12, 42 may include the first and second slot patterns 17, 47. This selective modification of the first and second tubes 12, 42 may be achieved by using laser micromachining to create a series of slots or notches in one side of each of the first and second tubes 12, 42, which may offset the neutral axis of each of the tubes 12, 42 towards one side of the respective tube (offset from, while remaining parallel to the respective first and second longitudinal axes 21, 23, and/or the centerline axis 100). In other embodiments, such selective modification is provided by selectively modifying the stiffness on one side of each of the first and second tubes 12, 42 to exhibit a more compliant behavior on that side which effectively shifts the neutral bending axis away from the centerline of each of the first and second tubes 12, 42 (for example, in polymer or braid reinforced tubes, this can be achieved by selectively applying a lower-durometer jacket material on the low-stiffness side). Such slot patterns may leave a “backbone” of material on each respective tube.

As mentioned above, the second tube 42 may be concentrically nested within the first tube 12. The second tube 42 may be disposed in the first tube 12 such that the first and second slot patterns 15, 45 overlap and are rotated 180 degrees with respect to each other, thereby positioning the modified neutral axes of the first and second tubes 12, 42 in diametric opposition. The first and second tubes 12, 42 may then be mechanically fixed to each other at a location distal to the slotted regions (e.g., at the first and second distal tips 16, 46). As shown with particular reference to FIG. 2, by linearly translating the first and second tubes 12, 42 with respect to each other at the first and second proximal ends 13, 43, the first and second distal ends 15, 45 of the first and second tubes 12, 42 bend bidirectionally about a singular plane (e.g., at the first bending joint 31), and thus may be considered to provide co-planar bending. This type of mechanism serves as the basis for the joints described in the present disclosure.

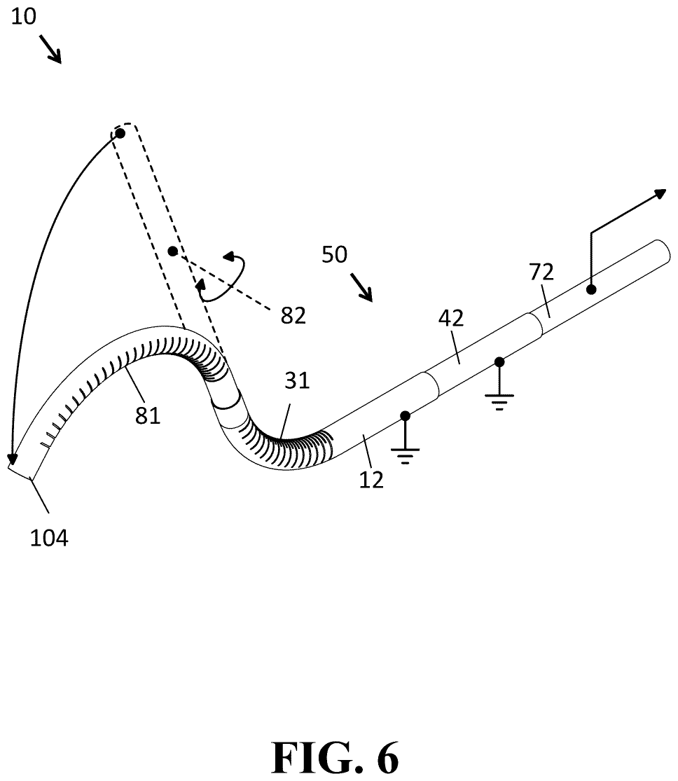

Referring now to FIGS. 3-6, the apparatus 10 is shown with three tubes (e.g., the first, second, and third tubes 12, 42, 72), according to some embodiments of the present disclosure. As discussed above with reference to FIG. 2, the first and second tubes 12, 42 may be assembled to provide the first bending joint 31. In further embodiments, and as described in greater detail below, the first, second, and third tubes 12, 42, 72 are assembled to create two serial bending joints (e.g., the first bending joint 31 and the second bending joint 81), where the first and second tubes 12, 42 are assembled to provide the first bending joint 31, and the second and third tubes 42, 72 are assembled to provide the second bending joint 81. The second bending joint 81 may be positioned distal relative to the first bending joint 31. In some embodiments, the first and second bending joints 31, 81 are axially spaced apart.

The first tube 12 may feature an outer diameter D1O and inner diameter D11. At the distal end 15 of the first tube 71, the first tube 12 may include the first slot pattern 17. In this sense, the first tube 12 may be configured with a singular bending section which is defined by an asymmetric stiffness profile which prescribes a primary bending plane. As suggested above, this selective weaking or asymmetric stiffness profile may be achieved by using laser micromanufacturing to create a series of slots or notches into one side of the first tube 12, thereby offsetting the first neutral axis 27 of the first tube 12 away from the first longitudinal axis 21 of the first tube 12. When combined with the complementary slot pattern 47 on the second tube 42, the assembly of the first and second tubes 12, 42 may create the first bending joint 31 via the agonist/antagonist methodology described previously, and as shown with reference to FIG. 2.

The second tube 42 may feature an outer diameter D2O and inner diameter D2I. Depending on the implementation, the second tube 42 must be sized to fit concentrically within the first tube 12. Thus, D2O may be less than D1I. As mentioned above, the second tube 42 may include the second slot pattern 47. In some embodiments, the second tube 42 further includes a fourth slot pattern (or, as mentioned above, a deflectable section) 48. The fourth slot pattern 48 may be positioned distally relative to the second slot pattern 47 (e.g., the fourth slot pattern 48 may be positioned closer to the distal tip 46 of the second tube 42 than the second slot pattern 47). Thus, the second tube 42 may include two discrete segments configured with the aforementioned asymmetric stiffness profile (in effect creating two serial bending sections via the second and fourth slot patterns 47, 48). The second slot pattern 47 of the second tube 42 may be aligned with the first slot pattern 17 on the first tube 12 in order to provide the first bending joint 31, as discussed above. In turn, the fourth slot pattern 48 of the second tube 42 may be aligned with a fifth slot pattern (or, as suggested above, a deflectable section) 78 of the third tube 72 in order to similarly provide the second bending joint 81. Accordingly, the second tube 42 may include the fourth deflectable section 48, the third tube 72 may include the fifth deflectable section 78, the fourth and fifth deflectable sections 48, 78 being selectively weakened portions of the second and third tubes 42, 72.

The third tube 72 may feature an outer diameter DO3 and an inner diameter DI3. Depending on the implementation, the third tube 72 must be sized to fit concentrically within the second tube 42. Thus, DO3 may be less than DI2. As mentioned above, the third tube 72 may include the third slot pattern 77 and the fifth slot pattern 78. The firth slot pattern 78 may be positioned distally relative to the third slot pattern 77 (e.g., the fifth slot pattern 78 may be positioned closer to the third distal tip 76 of the third tube 72 than the second slot pattern 77). In some embodiments, the fifth slot pattern 78 incorporates the aforementioned asymmetric stiffness profile, while the third slot pattern 77 incorporates an omnidirectionally compliant flexible section with high torsional stiffness. As suggested above, the fifth slot pattern 78 of the third tube 72 may be aligned with the fourth slot pattern 48 on the second tube 42 to define the second bending joint 81. In turn, the third slot pattern 77 of the third tube 72 may be aligned with the first and second slot patterns 12, 47 of the first and second tubes 12, 42 in order to reduce the stiffness of the third tube 72 in that region, in order to permit the actuation of the first bending joint 31. This longitudinal region defined by the first, second, and third slot patterns 17, 47, 77 may be designed such that its flexural stiffness is substantially less than the flexural stiffness longitudinal region defined by the fourth and fifth slot patterns 48, 78 that form the first bending joint 31.

As suggested above, the first, second, and third tubes 12, 42, 72 may be concentrically nested and mechanically joined at specific locations along the structure to provide the multi-jointed manipulator system discussed herein. As a first example, the first and second tubes 12, 42 may be joined at a location distal to the first and second deflectable sections 17, 47. As a second example, the second and third tubes 42, 72 may be joined at a location distal to the fourth and fifth deflectable sections 48, 78.

As suggested above, the interaction between the mechanics of the second and third tubes 42, 72 may form the second bending joint 81. In some embodiments, the stiffness of the third tube 72 does not contribute substantially to the combined mechanics of the first and second tubes 12, 42. For example, the third slot pattern 77 may provide a passively compliant region that overlaps with the longitudinal region formed by the first bending joint 31 formed by the first and second tubes 12, 42.

During assembly, the second tube 42 may be concentrically disposed within the third tube 72, and may be axially positioned such that the second slot pattern 47 of the second tube 42 and the first slot pattern 17 of the first tube 12 overlap, with the first and second neutral axes 27, 29 of the first and second tubes 12, 42 in opposition with each other in an agonist/antagonist configuration. The third tube 72 may then be joined (e.g., welded) to the second tube 42 at a location distal relative to the second slot pattern of the second tube 42, but proximal relative to the fourth slot pattern 48 of the second tube 42, as shown with reference to FIG. 4.

In some embodiments, and as suggested above, the interaction between the mechanics of the second and third tubes 42, 72 provides the second bending joint 81. During assembly, the third tube 72 may be concentrically disposed within the second tube 42, and is axially positioned such that the fourth slot pattern 48 of the second tube 42 and the fifth slot pattern 78 of the third tube 72 overlap, such that the second and third neutral axes 29, 33 of the second and third tubes 42, 72 are in opposition with each other in an agonist/antagonist configuration. The second tube 42 may then be joined to the third tube 72 at a location distal relative to the fourth and fifth slot patterns 48, 78 (e.g., the distal bending sections of both tubes), as shown with particular reference to FIG. 4. The third slot pattern 77 of the third tube 72 may thus form a passively compliant segment that overlaps with the first bending joint 31 provided by the interaction between the first and second tubes 12, 42.

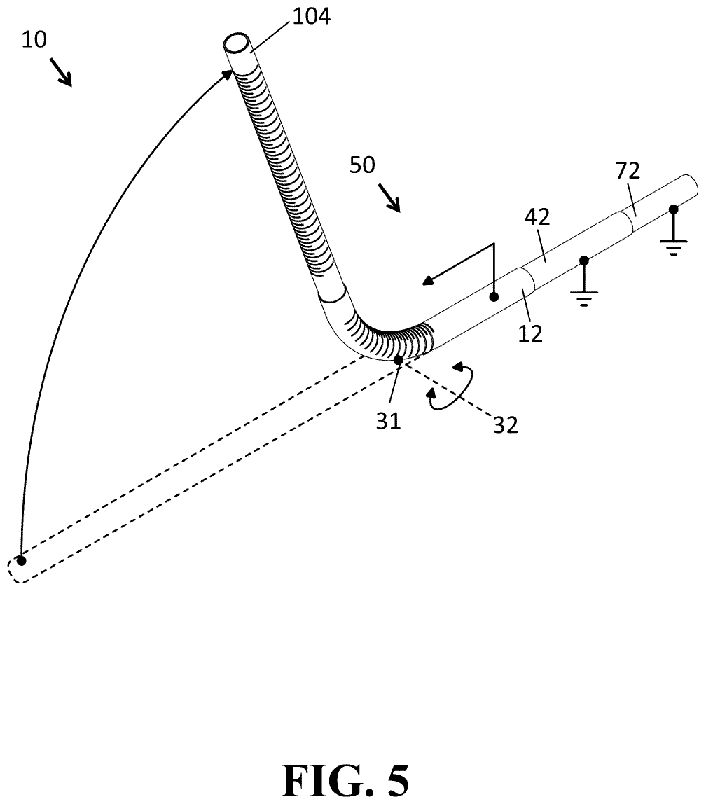

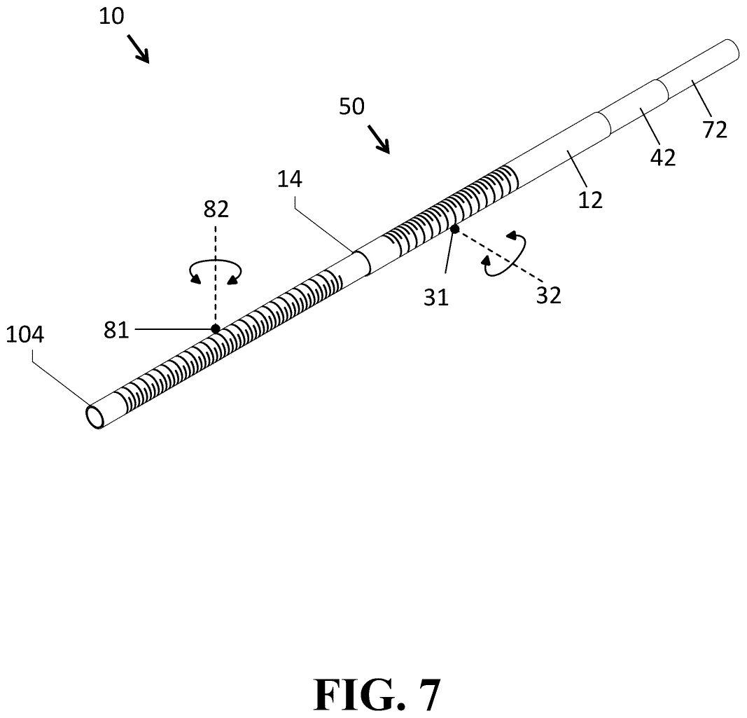

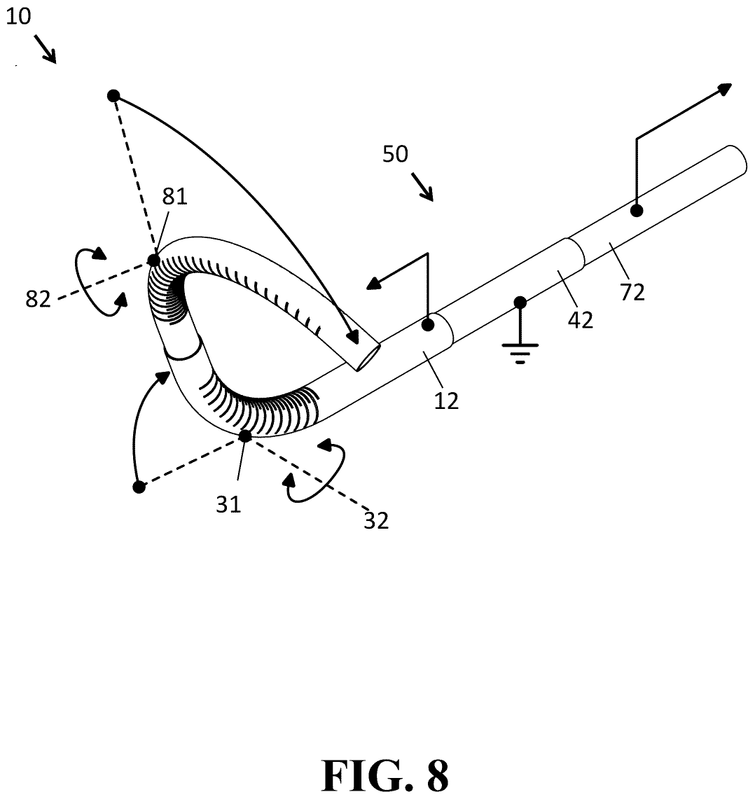

Referring now to FIGS. 7 and 8, the apparatus 10 is shown being actuated to bend in non-coplanar directions, according to some embodiments of the present disclosure. As shown with reference to FIG. 4, the axes of the first and second bending joints 31, 81 may be situated in parallel, and the resulting joint motions may be coplanar (e.g., the bends formed by the first and second bending joints 31, 81 may be coplanar all motion of the apparatus 10 occurs about a single plane), as shown with additional reference to FIG. 5. In further embodiments, and as shown, the first and second bending joints 31, 81 may have different orientations. For example, and as shown with particular reference to FIG. 7, the second bending joint 81 may be oriented orthogonal relative to the first bending joint 31, thus allowing the second bending joint 81 to be manipulated out of plane with respect to the first bending joint 31, as shown with particular reference to FIG. 8. Angles between the first and second joint axes 32, 82 may be made arbitrary by adjusting the rotational orientation of the aforementioned slot patterns of subsequent joints. Further, the total angle of deflection and curvature of subsequent joints can be made arbitrary by modifying a number of parameters used to define the slots that comprise the agonist/antagonist segments, including pitch, slot width, overlap width/angle, total pattern width/angle, etc.

Accordingly, in some embodiments, the tube assembly 50 is actuable to form the first bend (e.g., at the first bending joint 31) and the second bend (e.g., at the second bending joint 81) in co-planar directions, and in other embodiments, the tube assembly 50 is actuable to form the first bend and the second bend in non-coplanar directions.

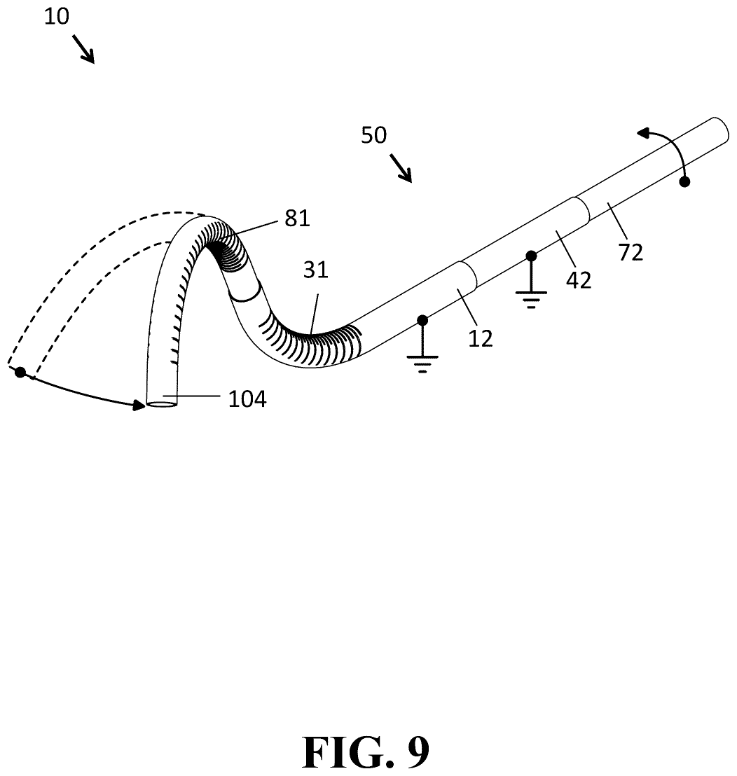

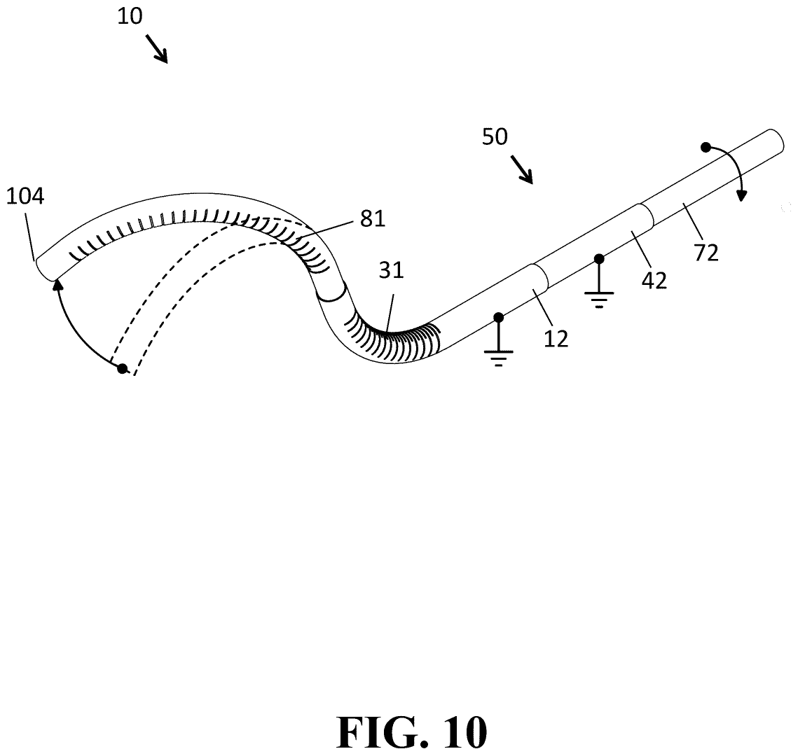

Referring now to FIGS. 9-12, the apparatus 10 is shown being actuated to provide a panning degree of freedom, according to some embodiments of the present disclosure. For instance, the apparatus 10 may be configured to exhibit a “panning” maneuver by twisting the third tube 72 with respect to the first and second tubes 12, 42, as shown in FIGS. 9-10, so long as the second and third distal sections 45, 75 of the second and third tubes 42, 72 are torsionally decoupled from the distal section 15 of the first tube 12. As a first example, when the third tube 72 is twisted, the higher stiffness backbones formed by the slot patterns of the first second joint 81 (e.g., the fourth and fifth slot patterns 48, 78) may take on a helical shape as prescribed by the torsional stiffness of the second and third tubes 42, 72 tubes in that region. As a result, when the second bending joint 81 is actuated, it may exhibit a corkscrew shape when actuated, effectively creating a lateral panning motion at the distal tip 104 of the apparatus 10. This panning motion may provide inherent coupling of all joints of the apparatus 10, but may be relatively decoupled by ensuring that the torsional stiffness of the panning joint (e.g., the second bending joint 81) is less than all other joints proximal to it (e.g., the first bending joint 31).

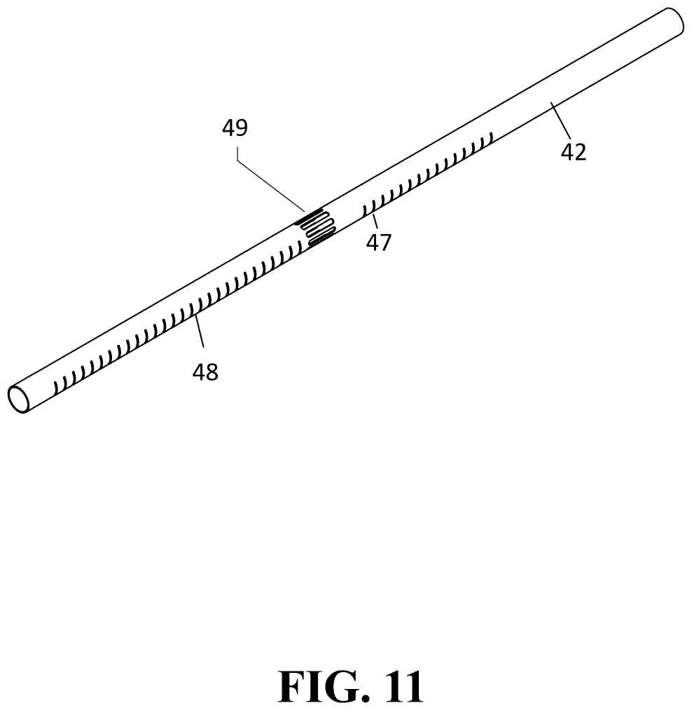

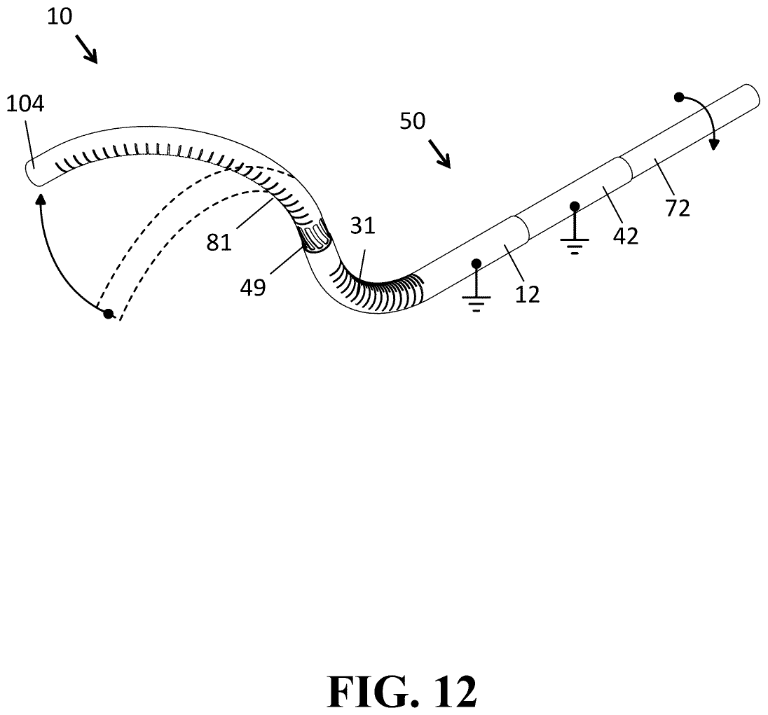

As a second example, and as shown with particular reference to FIGS. 11 and 12, the second tube 42 may include a short torsional decoupling segment 49 between first and second bending joints 31, 81, which exhibits high axial stiffness and low torsional stiffness. Depending on the implementation, the decoupling segment 49 may be axially located in between the second and fourth slot patterns 47, 48 of the second tube 42.

In some embodiments, the decoupling segment 49 includes several axial slots machined into the second tube 42 which locally weakens the second tube 42 tube torsionally, while maintaining high axial stiffness. In other words, the apparatus 10 may further include the decoupling segment 49 disposed on the second tube 42. For example, while the first and second slot patterns 47, 48 are generally depicted herein as spanning circumferentially around the second tube 42, the axial slots forming the decoupling segment 49 may extend parallel (or substantially parallel) to the second longitudinal axis 23 depicted with additional reference to FIG. 3.

In some embodiments, the decoupling segment 49 of the second tube 42 increases the range of the aforementioned panning degree of freedom, in order to maintain better alignment of the second and third tubes 42, 72, and maintain better circularity of the second and third distal segments 45, 75 (instead of helicity). For instance, the decoupling segment 49 may ensure that the twist imparted by the third tube 72 is primarily imparted on the decoupling segment 49 instead of being distributed along the second and third tubes 42, 72.

Depending on the implementation, the aforementioned panning degree of freedom (provided by the second bending joint 81) relies on the first bending joint 31 to be in an already-actuated configuration, as shown in FIGS. 9 and 10. Further, because the realized shape is helical (although the degree of helical deflection can be mitigated through the incorporation of the decoupling segment 49 as discussed previously), a rolling motion about the axis of the second bending joint 81 may be created as well. In this sense, twisting the third tube 72 with the first bending joint 31 in an unactuated configuration may result in a pure roll at the distal tip 104.

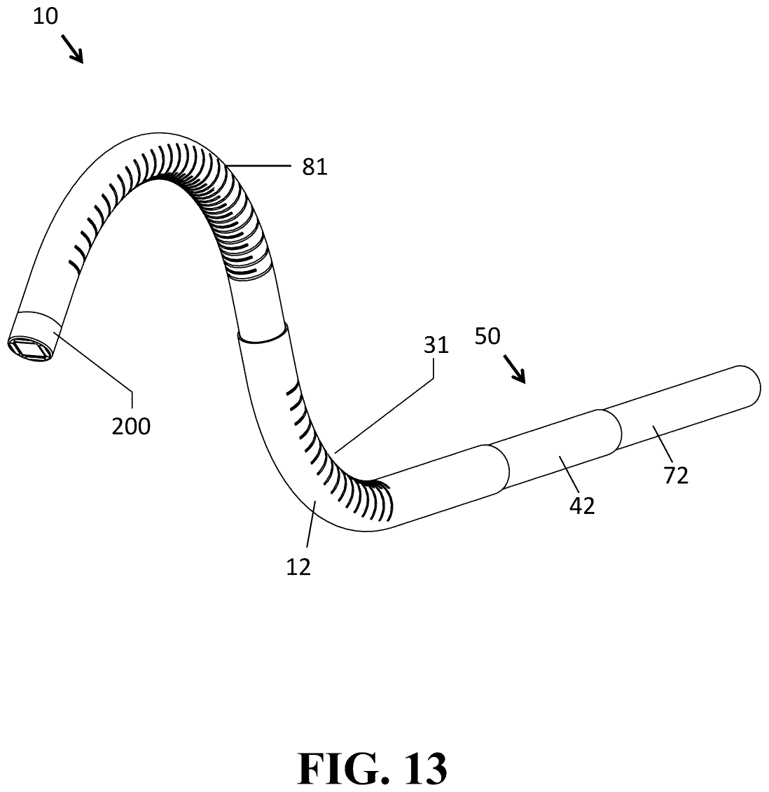

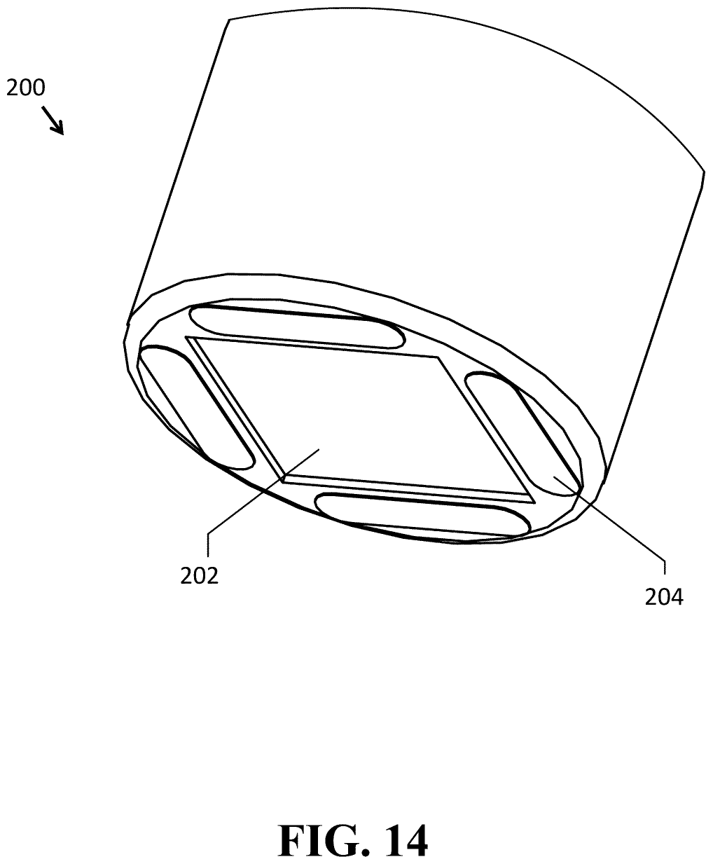

Referring now to FIGS. 13 and 14, the apparatus 10 is shown with a videoscope, according to some embodiments of the present disclosure. In some embodiments, apparatus 10 is actuated to generate a roll motion about the axis of the second bending joint 81 (e.g., about the second joint axis 82). In such embodiments where the apparatus 10 includes a videoscope 200 on the distal tip 104 of the apparatus 10 as shown with refence to FIG. 13, such a motion may also redirect the camera view of the videoscope 200 laterally, similar to the panning degree of freedom discussed above. However, the center of view may still intersect the axis of the videoscope 200 but from a different angle. Such a degree of freedom may be exploited in lieu of the panning degree of freedom to provide visualization of areas on either side of the straight view of the videoscope 200. Thus, the apparatus 10 may further include the videoscope 200 disposed on a distal tip of the tube assembly 50.

In further embodiments, the actuation roles of the first and second tubes 12, 72 as discussed above may be reversed. For instance, in this variation, the third tube 72 may be the shortest tube (as opposed to the first tube 12), having a single bending segment with an asymmetric stiffness profile as discussed above, and has its distal tip 76 attached to the second tube 42 at the end of the second bending joint 81. In such variations, the second tube 42 may define the same profile as described above. In such variations, the third tube 72 may define one distal bending segment with asymmetric stiffness profile, and a proximal segment incorporating an omnidirectionally compliant flexible section. The first and second tubes 12, 42 may thus be attached at the end of the distal bending segment. The first bending joint 31 may then be actuated by relative translation of the second and third tubes 42, 72, while the second bending joint 81 may be actuated by relative translation of the first and second tubes 12, 42, and the panning degree of freedom may thus be actuated by twisting the first tube 12 at its base. This variation may further increase the working range of the panning degree of freedom (since it is driven by the stiffer outer tube) and increase overall manipulator stiffness.

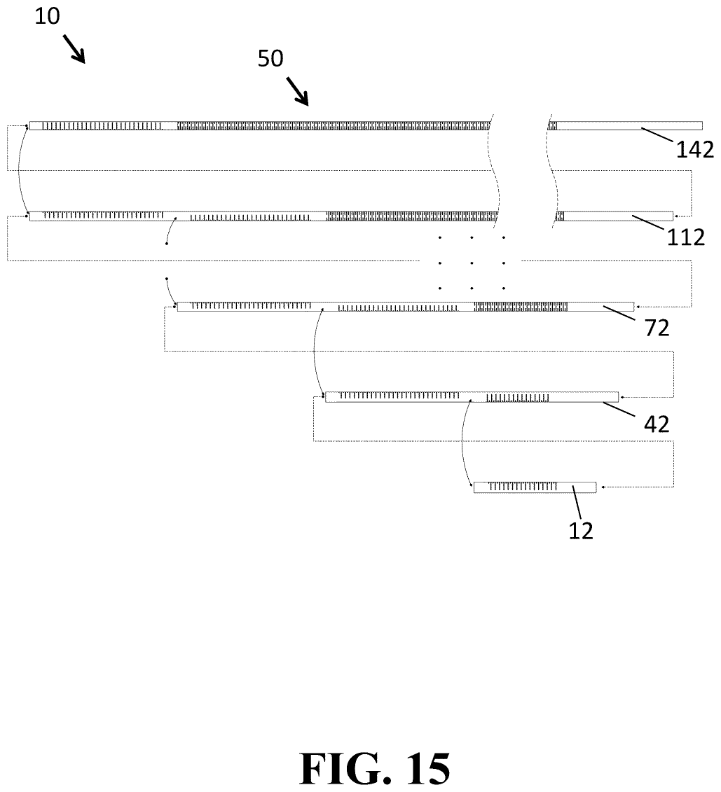

Referring now to FIG. 15, the apparatus 10 is shown with five tubes, according to some embodiments of the present disclosure. For instance, in addition to the first, second, and third tubes 12, 42, and 72, the apparatus 10 may include any number of additional tubes, including a fourth tube 112 and a fifth tube 142. In this sense, the general manipulator scheme of the apparatus 10 can be generalized to N tubes to create J independently-actuatable bending degrees of freedom (e.g., joints), where J=N-1 bending degrees of freedom. Thus we may refer to any arbitrary nth tube such that n∈{1, . . . , N}, and any arbitrary jth joint such that j∈{1, . . . , J}. If we consider tube n=1 to be the first tube 12, and tube n=N to be the inner-most tube, general design guidelines are as follows.

For tube (n=1), i.e. for the outer-most tube, there is a singular steering section, the location and orientation of which defines the axis of joint j=1. For tube (n=2), there are two steering sections, the first of which overlaps the distal-most steering section of the (n-1)th tube to complete the agonist/antagonist bending scheme for joint j=n-1, and the second of which defines the orientation and location of joint j=n and sets up the agonist/antagonist bending scheme when combined with the proximal-most joint of the next tube. For any nth tube such that 2<n<N, there are 2 steering sections which follow the same logic as above (i.e. the proximal-most steering section overlaps with the distal-most steering section of the previous tube to complete joint j=n-1, and the distal-most steering section defines the location and orientation of the next joint j=n), and a passive bending section overlaps with all of the steering sections which define joints from (1≤j≤n-1). Note that for all tubes n>1, tube nis welded to tube n-1 at a location distal to joint j=n-1. All tubes must nest concentrically, so the inner diameter of the nth tube may be greater than the outer diameter of the relative (nth-1) tube. For further generalizability, the axes orientation of each joint may be arbitrary (i.e. the axes do not need to be parallel). In the alternative tube order embodiment mentioned above, the logic may be the same, however the order of tubes may be reversed (e.g., n=1 corresponds to the inner-most tube, n=N corresponds to the outer-most tube).

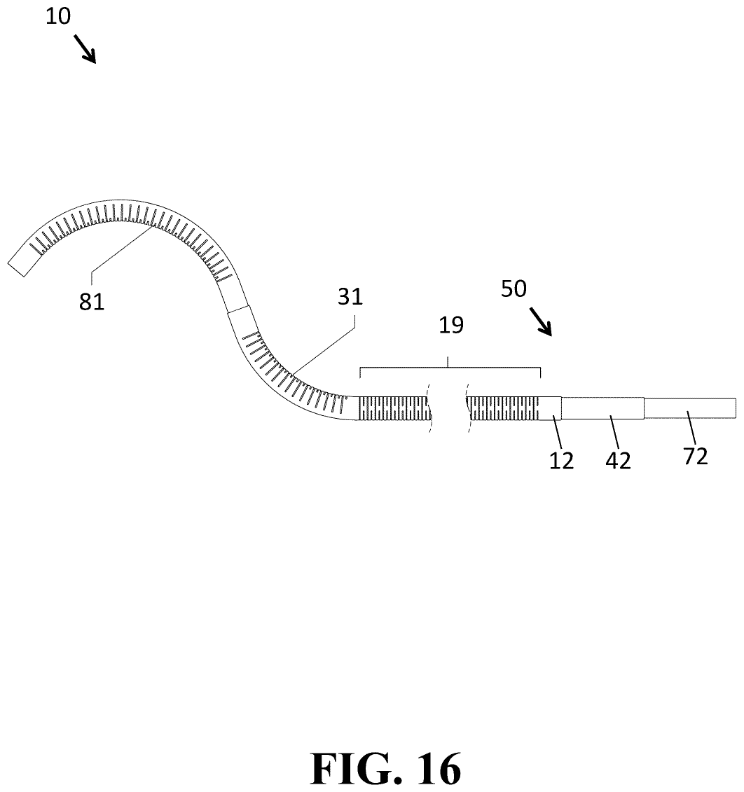

Referring to FIG. 16, the apparatus 10 is shown with a long proximal flexible section (e.g., transmission section) 19, according to some embodiments of the present disclosure. For instance, in applications where the apparatus 10 is required to passively navigate along a tortuous path, including applications in flexible endoscopy, the tubes comprising the apparatus 10 may be configured to exhibit this passive flexibility along the length. For example, all tubes comprising the apparatus 10 (e.g., the first, second, and third tubes 12, 42, 72) may be configured with an omnidirectionally compliant transmission section 19 proximal to the proximal-most joint (e.g., the first and second bending joints 31, 81, as applicable). As shown, this can be achieved by laser machining a series of slots in the tube which reduces the local stiffness of the tube while keeping the neutral axis colinear with the tube's geometric axis. In other embodiments, this transmission section 19 may be realized by attaching the steering sections of the tubes to low-stiffness polymeric tubes which may or may not feature braid reinforcement.

In some embodiments, apparatus 10 features an internal liner (i.e. PTFE) for creating a lubricious surface on the inner diameter to facilitate the passage of other tools and devices. The apparatus 10 may also feature an external polymeric jacket (i.e. PEBAX, Nylon12, Vitron Rubber, FEP, PET), with an optional hydrophilic coating, for the purpose of adding lubricity to the outer diameter of the system to facilitate passage of the system through a natural lumen or the working channel of an endoscope.

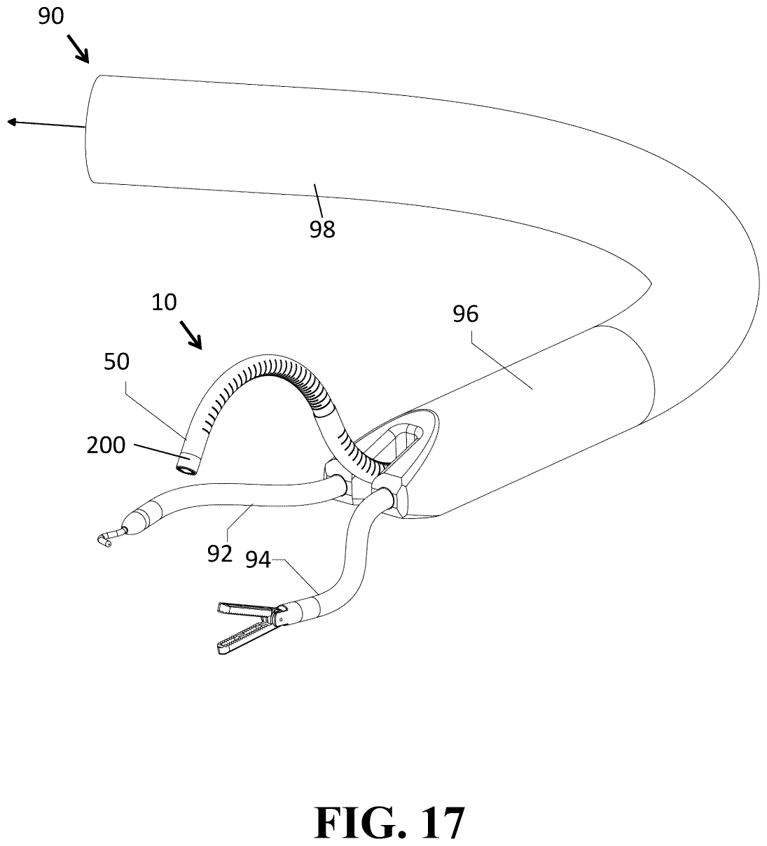

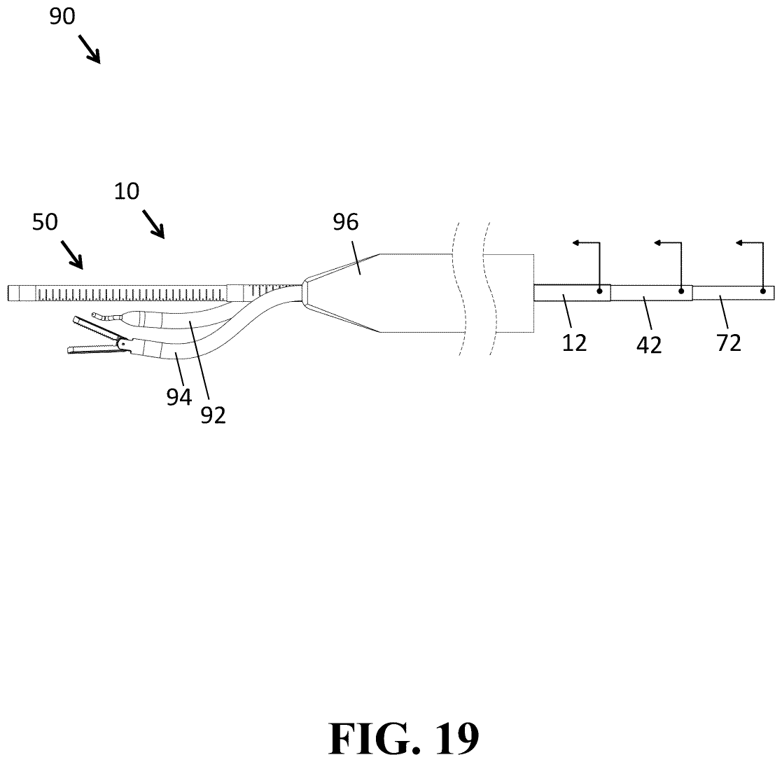

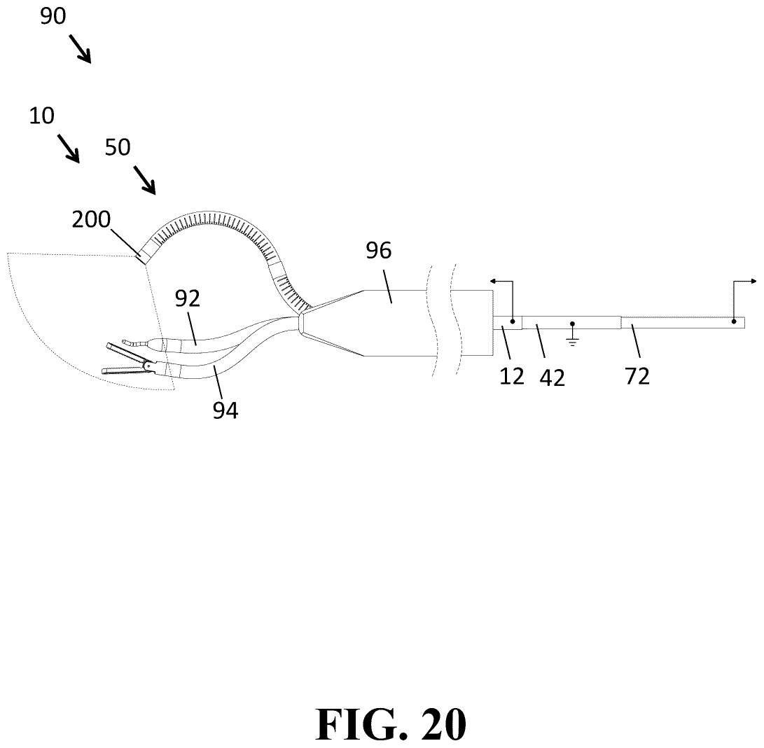

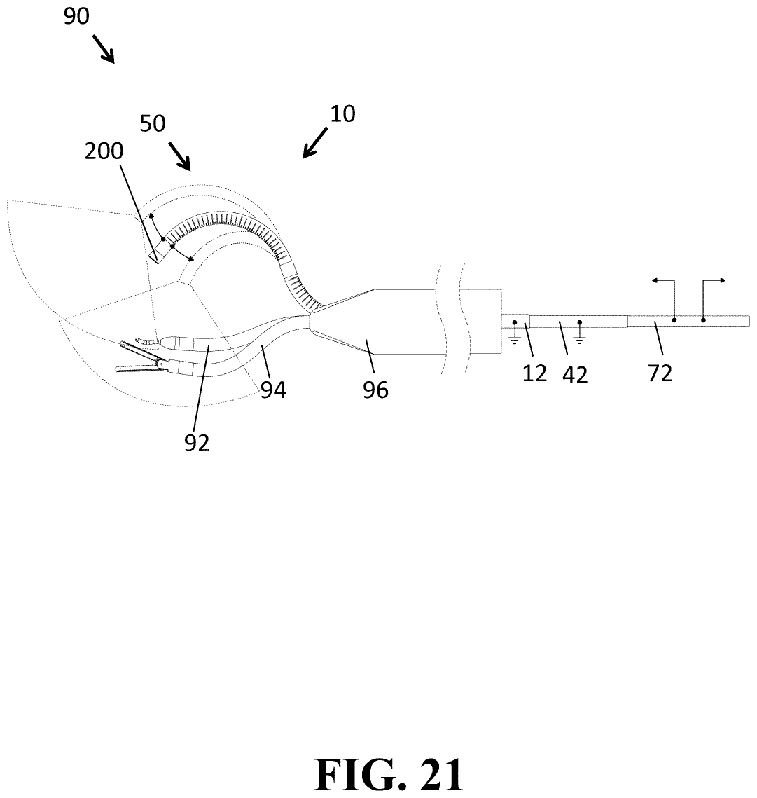

Referring to FIGS. 17-21, a robotic videoscope system 90 is shown, according to some embodiments of the present disclosure. For example, the present disclosure provides for an integrated imaging and illumination system 90 for the purpose of providing illumination and visualization in rigid and flexible medical robotic systems. Such an embodiment may be used as part of a larger rigid or flexible robotic system to provide lighting and visualization of the surgical site. The ability to separately elevate, tilt, and pan the imaging system, in combination with separate interventional tool arms 92 and 94, may be useful for mimicking the biomechanics of a human head, neck, and arms in a surgical robotic system. As shown, the system 90 may include the apparatus 10 projecting from an endcap 96, along with the instrument manipulators 92, 94 disposed on the endcap 96. The endcap 96 may feature the videoscope 200 which, in some embodiments, includes an integrated complementary metal oxide semiconductor (CMOS) camera sensor 202 and an illumination source (e.g., an array of fiber optic or LED illumination sources) 204 (as shown with additional reference to FIG. 14). The system 90 may further include tube manipulators. The system 90 may optionally be additional working channels for the passage of tools or irrigation fluid. The system 90 may be delivered through a separate rigid or flexible overtube 98, and may be combined with one or more separate manually or robotically-controlled instrument manipulators to realize a surgical robotic system.

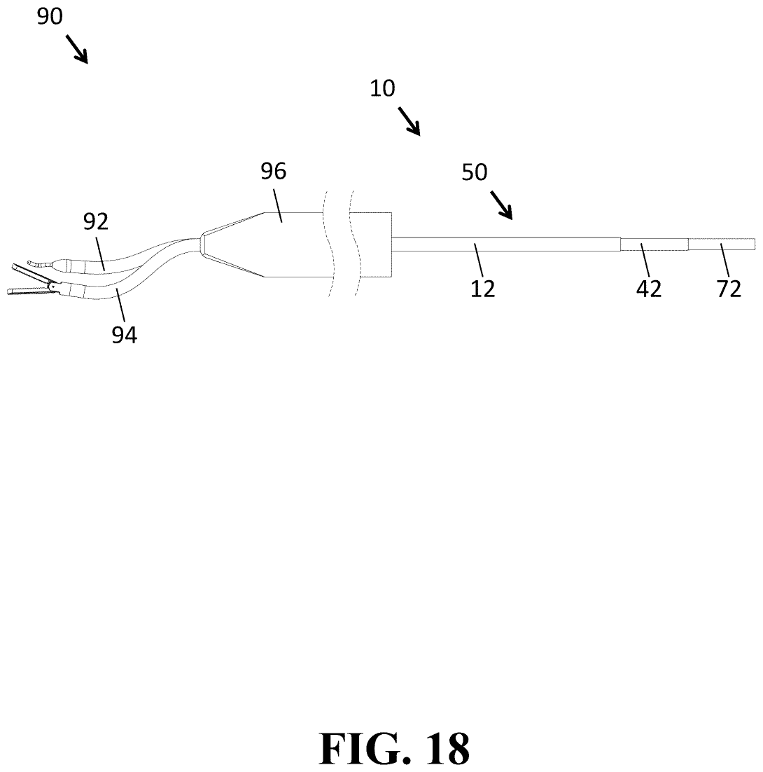

Accordingly, the system 90 may be used to realize a robotically-controlled video-endoscope capable of separate elevation, tilt, and pan degrees of freedom. As shown with reference to FIG. 18, the system 90 may be deployed by translating the bases of the first, second, and third tubes 12, 42, 72 linearly at the same rate, and the system 90 may be linearly inserted into the surgical field. By mechanically fixing the second tube 42, pulling on the third tube 72, and pushing on the first tube 12, the elevation and tilt degrees of freedom may be actuated (respectively), thereby bringing the instrument manipulators 92, 94 into view. For further tilt adjustment, the first and second tubes 12, 42 tubes may be mechanically fixed, and the third tube 72 may be translated.

As mentioned previously, rotating the third tube 72 can cause the videoscope 200 to exhibit a panning motion for looking left and right. This collection of actuatable degrees-of-freedom can be exploited to provide comprehensive, on-demand visual coverage of the surgical field during complex interventional procedures. In the context of a flexible robotic system, the tubes comprising the system 90 should feature the passively compliant flexible transmission discussed with reference to FIG. 16, where the transmission is designed with the general design goal of maximizing axial and torsional stiffness (to ensure that adequate force and torque can be transmitted to the distal jointed segments to actuate the elevate/tilt/pan degrees of freedom) while minimizing flexural stiffness (to ensure that the system 90 is capable of navigating the tortuous anatomy through which it may be passed).

In accordance with the discussion of the apparatus 10 and the system 90 herein, the present disclosure further provides for a method of performing endoscopic surgery or other boroscopic operations. The method may include providing the tube assembly 50, the tube assembly 50 including the first tube 12, the second tube 42 concentrically nested within the first tube 12, and the third tube 72 concentrically nested within the second tube 42. The method may further include forming the first bend (at the first bending joint 31) by relative axial translation between the first tube 12 and the second tube 42, and forming the second bend (at the second bending joint 81) by relative axial transaction between the second tube 42 and the third tube 72. The method may further include providing the videoscope 200 disposed on a distal tip of the tube assembly 50.

In the drawings, not all reference numbers are included in each drawing, for the sake of clarity. In addition, positional terms such as “upper,” “lower,” “side,” “top,” “bottom,” etc. refer to the apparatus when in the orientation shown in the drawing, or as otherwise described. A person of skill in the art will recognize that the apparatus can assume different orientations when in use.

Thus, although there have been described particular embodiments of the present invention of a new and useful APPARATUSES AND METHODS FOR A TUBULAR CONCENTRIC TUBE CONTINUUM MANIPULATOR, it is not intended that such references to particular embodiments be construed as limitations upon the scope of this invention.

What is claimed is:

1. A visual inspection apparatus, comprising:
a tube assembly including a first tube, a second tube concentrically nested within the first tube, and a third tube concentrically nested within the second tube,
wherein the first tube includes a first deflectable section, the second tube includes a second deflectable section, the third tube includes a third deflectable section, the first, second, and third deflectable sections being selectively weakened portions of the first, second, and third tubes,
wherein the first and second tubes are joined at a location distal to the first and second deflectable sections,
wherein the second tube includes a fourth deflectable section, the third tube includes a fifth deflectable section, the fourth and fifth deflectable sections being selectively weakened portions of the second and third tubes,
wherein the second and third tubes are joined at a location distal to the fourth and fifth deflectable sections,
wherein the tube assembly is actuable to form a first bend by relative axial translation between the first tube and the second tube, and
wherein the tube assembly is actuable to form a second bend by relative axial transaction between the second tube and the third tube.

2. The apparatus of claim 1, further comprising a videoscope disposed on a distal tip of the tube assembly.

3. The apparatus of claim 1, wherein the videoscope includes a CMOS sensor and an illumination source.

4. The apparatus of claim 1, wherein the tube assembly is actuable to form the first bend and the second bend in co-planar directions.

5. The apparatus of claim 1, wherein the tube assembly is actuable to form the first bend and the second bend in non-coplanar directions.

6. The apparatus of claim 1, further comprising a decoupling segment disposed on the second tube.

7. A visual inspection system, comprising:
an endcap;
one or more instrument manipulators disposed on the endcap; and
a tube assembly disposed on the endcap, the tube assembly including a first tube, a second tube concentrically nested within the first tube, and a third tube concentrically nested within the second tube,
wherein the first tube includes a first deflectable section, the second tube includes a second deflectable section, the third tube includes a third deflectable section, the first, second, and third deflectable sections being selectively weakened portions of the first, second, and third tubes,
wherein the first and second tubes are joined at a location distal to the first and second deflectable sections,
wherein the second tube includes a fourth deflectable section, the third tube includes a fifth deflectable section, the fourth and fifth deflectable sections being selectively weakened portions of the second and third tubes,
wherein the second and third tubes are joined at a location distal to the fourth and fifth deflectable sections,
wherein the tube assembly is actuable to form a first bend by relative axial translation between the first tube and the second tube, and
wherein the tube assembly is actuable to form a second bend by relative axial transaction between the second tube and the third tube.

8. The apparatus of claim 7, further comprising a videoscope disposed on a distal tip of the tube assembly.

9. The apparatus of claim 8, wherein the videoscope includes a CMOS sensor and an illumination source.

10. The apparatus of claim 7, wherein the tube assembly is actuable to form the first bend and the second bend in co-planar directions.

11. The apparatus of claim 7, wherein the tube assembly is actuable to form the first bend and the second bend in non-coplanar directions.

12. The apparatus of claim 7, further comprising a decoupling segment disposed on the second tube.

13. A method, comprising:
providing a tube assembly, the tube assembly including a first tube, a second tube concentrically nested within the first tube, and a third tube concentrically nested within the second tube;
forming a first bend by relative axial translation between the first tube and the second tube; and
forming a second bend by relative axial transaction between the second tube and the third tube,
wherein the first tube includes a first deflectable section, the second tube includes a second deflectable section, the third tube includes a third deflectable section, the first, second, and third deflectable sections being selectively weakened portions of the first, second, and third tubes,
wherein the first and second tubes are joined at a location distal to the first and second deflectable sections,
wherein the second tube includes a fourth deflectable section, the third tube includes a fifth deflectable section, the fourth and fifth deflectable sections being selectively weakened portions of the second and third tubes,
wherein the second and third tubes are joined at a location distal to the fourth and fifth deflectable sections.

14. The method of claim 13, further comprising providing a videoscope disposed on a distal tip of the tube assembly.

15. The method of claim 14, wherein the videoscope includes a CMOS sensor and an illumination source.

16. The method of claim 13, wherein the first bend and the second bend are formed in co-planar directions.

17. The method of claim 13, wherein the first bend and the second bend are formed in non-co-planar directions.

18. The method of claim 13, wherein the second tube includes a decoupling segment.
